(19) United States
(12) Patent Application Publication
(10) Pub. No.: US 2011/0275103 A1
(43) Pub. Date: Nov. 10, 2011

(54) EMISSION RATIOMETRIC REPORTERS OF MEMBRANE PDK1 ACTIVATION

(75) Inventors: Jin Zhang, Rosedale, MD (US); Xinxin Gao, Baltimore, MD (US)

(73) Assignee: THE JOHNS HOPKINS UNIVERSITY, Baltimore, MD (US)

(21) Appl. No.: 13/096,689

(22) Filed: Apr. 28, 2011

Related U.S. Application Data
(60) Provisional application No. 61/328,718, filed on Apr. 28, 2010.

(51) Int. Cl. C12Q 1/48; C12N 15/62; B65D 85/84; C12N 1/21; C12N 1/19; C12N 5/10; C12N 11/02; C12N 15/63

(52) U.S. Cl. 435/15; 435/177; 536/23.2; 435/320.1; 435/252.3; 435/254.2; 435/419; 435/325; 435/366; 206/524.6

(57) ABSTRACT

Phosphoinositide-dependent kinase 1 (PDK1) activity reporters can be used in high throughput assays for drug screening.

This application claims the benefit of and incorporates by reference Ser. No. 61/328,718 filed on Apr. 28, 2010.

Work described in this specification was funded by National Institutes of Health grants DK-973368, CA-122673, and DK-084171. The U.S. government therefore has certain rights in the invention.

This application incorporates by reference the contents of a 24.5 kb text file created on Apr. 27, 2011 and named “sequencelisting.txt,” which is the sequence listing for this application.

### FIELD OF THE INVENTION

The invention relates to reporters for kinase activation.

### BACKGROUND

Phosphoinositide 3-kinase (PI3K)/Akt signaling plays a major role in cell metabolism, growth, and apoptosis (1). Defects in PI3K/Akt signaling have been implicated in many diseases, including cancer (2) and type 2 diabetes (3). The activation of this pathway is initiated at the plasma membrane, where phosphatidylinositol (3,4,5) trisphosphate (PI(3,4,5)P₃) generated by PI3K and degraded by PTEN (phosphatase and tensin homolog deleted on chromosome 10) recruits Akt to the membrane. Once at the membrane, Akt is phosphorylated in its activation loop by phosphoinositide-dependent kinase 1 (PDK1) and its hydrophobic motif by mammalian TOR complex 2 (4). Following these two phosphorylation events, Akt adopts an active conformation and proceeds to phosphorylate a variety of protein substrates involved in diverse cellular processes.

Although the activation of the PI3K/Akt signaling pathway has been extensively studied, the mechanisms by which several critical steps are regulated in the cell are still not well understood (5). For instance, it remains unclear whether growth factor stimulation leads to activation of PDK1. A better understanding of the complex cellular regulation of the PI3K/Akt signaling may require dissecting these molecular events in their specific cellular contexts. The plasma membrane is the site of activation of the PI3K/Akt pathway, and plasma membrane microdomains, such as sphingolipid- and cholesterol-enriched membrane rafts (6), emerge as important regulators of this pathway.

A recent study using fluorescence correlation spectroscopy (FCS) has indicated that raft microdomains play important roles in recruiting Akt to the membrane after PI(3,4,5)P₃ production (7). Disruption of membrane rafts inhibits the recruitment process (7). In addition, we have previously observed a preferential activation of Akt in membrane rafts using a fluorescence resonance energy transfer (FRET)-based Akt activity reporter (AktAR) (8). However, the molecular mechanisms by which membrane rafts control Akt recruitment and regulate its activation are yet to be determined. Furthermore, while spatial compartmentalization is believed to be a key mechanism for achieving specificity and efficiency in general (9), the role of membrane microdomain compartmentalization in PI3K/Akt signaling remains poorly defined.

### BRIEF DESCRIPTION OF THE DRAWINGS

FIGS. 1A-1F. Development and characterization of PDK1 activation reporters. FIG. 1A, a PDK1 activation reporter (PARE; SEQ ID NO:14, which can be encoded by SEQ ID NO:15) was generated with full-length PDK1 sandwiched by a pair of fluorescent proteins, ECFP and Citrine. PARE was targeted to membrane rafts using a targeting motif derived from Lyn kinase and to non-raft regions of the plasma membrane using a targeting motif derived from Kras. FIG. 1B, the localization of Lyn-PARE and PARE-Kras was confirmed by sucrose density gradient fractionation. Total cell lysates from HEK 293 cells overexpressing Lyn-PARE or PARE-Kras were subjected to sucrose density gradient fractionation, followed by Western blotting. Cholera toxin subunit B was used as a raft marker. The reporters were detected with an anti-GFP antibody. FIG. 1C, representative time courses showing the responses of PARE (n=5), Lyn-PARE (n=5), and PARE-Kras (n=4) in serum-starved NIH 3T3 cells after stimulation with 100 μM PV. FIG. 1D, pseudocolor images showing the response of Lyn-PARE to 50 ng/ml PDGF in NIH3T3 cells. The yellow fluorescence image (“YFP,” left) shows the localization of Lyn-PARE. FIG. 1E, pseudocolor images showing the response of PARE-Kras to 50 ng/ml PDGF in NIH 3T3 cells. The yellow fluorescence image (“YFP,” left) shows the localization of PARE-Kras. FIG. 1F, stimulation of serum-starved NIH 3T3 cells with 50 ng/ml PDGF induced an emission ratio change in Lyn-PARE (n=7), but not PARE (n=5) or PARE-Kras (n=6).

FIGS. 2A-2B. PTEN is primarily localized to non-raft regions of the plasma membrane. FIG. 2A, crude plasma membranes from HEK 293 cells were solubilized and subjected to sucrose density gradient fractionation, followed by Western blotting with an anti-PTEN antibody. Cholera toxin subunit B was used as a raft marker. Anti-tubulin was used to ensure the separation of membrane proteins from cytosolic proteins. FIG. 2B, statistically significant differences between PTEN levels in rafts and non-raft regions (p<0.001, n=3). Densitometric analysis indicated that the majority of the membrane PTEN resides in non-raft regions.

FIGS. 3A-3E. Genetic targeting of PTEN to membrane rafts abolishes PDK1 activation, Akt membrane recruitment, and Akt activity. FIG. 3A, PTEN A4, fused with a C-terminal fluorescent protein (FP), was targeted to membrane rafts and non-raft regions with a Lyn or Kras motif. FIG. 3B, the membrane localization of Lyn-PTEN A4 or PTEN A4-Kras was verified by sucrose density gradient fractionation of total cell lysates of HEK 293 cells overexpressing Lyn-PTEN A4-YFP or PTEN A4-YFP-Kras. Cholera toxin subunit B was used as a raft marker. Targeted PTEN A4 was detected with an anti-GFP antibody. Fluorescence images show the membrane localization of PTEN. FIG. 3C, representative time courses indicating that the response of Lyn-PARE was abolished by Lyn-PTEN A4 (n=5, PTEN fused with mCherry), but not PTEN A4-Kras (n=7). FIG. 3D, representative time courses demonstrating that the membrane translocation of the AktPH domain was abolished by Lyn-PTEN A4 (n=4, PTEN fused with mCherry), but not PTEN A4-Kras (n=4). FIG. 3E, representative time courses indicating that the response of AktAR was abolished by Lyn-PTEN A4 (n=7, PTEN fused with mCherry), but not PTEN A4-Kras (n=6).

FIGS. 4A-4F. Ceramide recruits PTEN to membrane rafts and suppresses PDK1 activation, Akt membrane recruitment, and Akt activity. FIG. 4A, ceramide recruits PTEN to membrane rafts. Crude plasma membranes from 3T3 L1 adipocytes (in the presence or absence of 50 μM C2-ceramide) were solubilized and subjected to sucrose density gradient fractionation, followed by Western blotting with an anti-PTEN antibody. Cholera toxin subunit B was used as a raft marker. Anti-tubulin was used to ensure the separation of membrane proteins from cytosolic proteins. FIG. 4B, densitometric analysis indicated a statistically significant difference between the raft PTEN levels in the presence of C2-ceramide and those in its absence (p<0.01, n=3). FIG. 4C, representative time courses showing the response of Lyn-PARE (n=7) in 3T3 L1 preadipocytes was abolished by pre-incubation with 50 μM C2-ceramide (n=6). FIG. 4D, representative time courses showing that membrane translocation of Akt PH domain (n=3) in 3T3 L1 preadipocytes was abolished by pre-incubation with 50 μM C2-ceramide (n=3). FIG. 4E, representative time courses showing that the response of AktAR (n=6) in 3T3 L1 preadipocytes was abolished by pre-incubation of 50 μM C2-ceramide (n=5). FIG. 4F, ceramide-mediated suppression of insulin-induced glucose uptake in 3T3 L1 adipocytes. Pre-incubation of 50 μM C2-ceramide with 3T3 L1 adipocytes for 60 min inhibited insulin-induced glucose uptake in these cells (p<0.0001, n=3).

FIGS. 5A-C. The response of Lyn-PARE is dependent on 3′ phosphoinositides. FIG. 5A, stimulation of serum starved HeLa cells with 100 ng/ml EGF induced an emission ratio change of Lyn-PARE (n=7), but not PARE (n=4) or PARE-Kras (n=7). Addition of 50 μM LY294002 reversed the response of Lyn-PARE (n=7). FIG. 5B, a representative time course shows the response of LynPARE in serum starved NIH 3T3 cells stimulated with 50 ng/ml PDGF, followed by addition of 50 μM LY294002 (n=3). Representative response of the Lyn-PARE (R4721474A) mutant is also shown (n=3). FIG. 5C, the response of Lyn-PARE to PV is dependent on 3′ phosphoinositides. A representative time course shows the response of Lyn-PARE to PV in serum starved NIH 3T3 cells, followed by 50 μM LY294002 treatment (n=5). Representative response of the Lyn-PARE (R4721474A) mutant is also shown (n=3).

FIGS. 6A-B. Phosphorylation of Akt by active PDK1 in membrane microdomains. FIG. 6A, crude plasma membranes from HEK 293 cells (stimulated with 400 nM insulin) were solubilized and subjected to sucrose density gradient fractionation followed by western analysis using an Akt or phospho-Akt (T308) antibody. Cholera toxin subunit B was used as a raft marker. Anti-tubulin was used to ensure the separation of membrane proteins from cytosolic proteins. For fractions 7, 8 and 9, ten times less volume was loaded. FIG. 6B, densitometric analysis of phospho-Akt normalized to total Akt showing a 5.0±2.5 fold (p<0.05, n=3) stronger signal of Akt phosphorylation (T308) in membrane rafts compared to non-rafts, suggesting that PDK1 is preferentially activated in membrane rafts.

FIGS. 7A-B. Genetic targeting of PTEN A4 to rafts, but not non-rafts, abolishes PDK1 activity. FIG. 7A, representative time courses showing responses of LynPARE in the presence of Lyn-PTEN A4 (n=5) or PTEN A4-Kras (n=7). Treatment with 50 mM H₂O₂ restored the response. FIG. 7B, PDGF induced responses (amplitude and t_{1/2} values) of Lyn-PARE in the presence or absence of Lyn-PTEN A4 or PTEN A4-Kras (**: p<0.01, ***: p<0.001).

FIGS. 8A-B. Genetic targeting of PTEN A4 to rafts, but not non-rafts, abolishes membrane recruitment of Akt. FIG. 8A, representative time courses showing membrane translocation of Akt PH domain in the presence of Lyn-PTEN A4 (n=4) or PTEN A4-Kras (n=4). Treatment with 50 mM H₂O₂ restored the membrane recruitment. FIG. 8B, PDGF induced translocation of Akt PH (response amplitude and t_{1/2} values) in the presence or absence of Lyn-PTEN A4 or PTEN A4-Kras (*: p<0.05, **: p<0.01, ***: p<0.001).

FIG. 9. Genetic targeting of PTEN A4 to rafts, but not non-rafts, abolishes Akt activity. PDGF induced responses (amplitude and t_{1/2} values) of AktAR in the presence or absence of Lyn-PTEN A4 or PTEN A4-Kras (**: p<0.01, ***: p<0.001).

FIG. 10. Distribution of Lyn-PARE, Akt PH domain and AktAR in the presence or absence of ceramide. Fluorescence images show the distribution of Lyn-PARE, Akt PH domain and AktAR in the presence or absence of 50 μM C2-ceramide.

FIGS. 11A-C. Ceramide suppresses PDK1 activation, membrane recruitment of Akt and Aid activity in NIH 3T3 cells. FIG. 11A, representative time courses showing the response of LynPARE (n=4) in NIH 3T3 cells was abolished with pre-incubation of 50 μM C2-ceramide for 60 min (n=9). FIG. 11B, representative time courses showing membrane translocation of Akt PH domain (n=4) in NIH 3T3 cells was abolished with pre-incubation of 50 μM C2-ceramide for 60 min (n=5). FIG. 11C, representative time courses showing the response of AktAR (n=4) in NIH 3T3 cells was abolished with pre-incubation of 50 μM C2-ceramide for 60 min (n=3).

FIGS. 12A-B. Compartmentalized PDK1 and PTEN activity in membrane microdomains is essential for proper PI3K/Akt signaling. FIG. 12A, preferential activation of Akt in rafts is mediated by raft-specific action of PDK1 and lack of PTEN-mediated down-regulation in these microdomains. FIG. 12B, dysregulation of the lipid raft-localized PI3K/Akt signaling as the underlying mechanism for insulin resistance. Ceramide may inhibit Akt signaling through promoting PKCζ phosphorylation of Akt or activating the Akt phosphatase PP2A. The ceramide-induced mis-localization of PTEN to membrane rafts critically contributes to the inhibitory effect of ceramide on PI3K/Akt signaling.

### DETAILED DESCRIPTION

This disclosure describes biosensors (also referred to as “activation reporters”) for PDK1 activation. PDK1 activation reporters are based on resonance energy transfer, particularly fluorescence resonance energy transfer (FRET). In particular, highly sensitive reporters molecules can be used to monitor temporal and spatial PDK1 activation in living tissues and cells, as well as in naturally occurring or synthetic lipid bilayers. PDK1 activation reporters are useful, inter alia, for high throughput drug screening and to monitor PDK1 activation in various disease states. As explained in more detail in the Examples below, in some embodiments PDK1 activation is monitored using specific and reversible genetically encoded FRET-based PDK1 activity reporters.

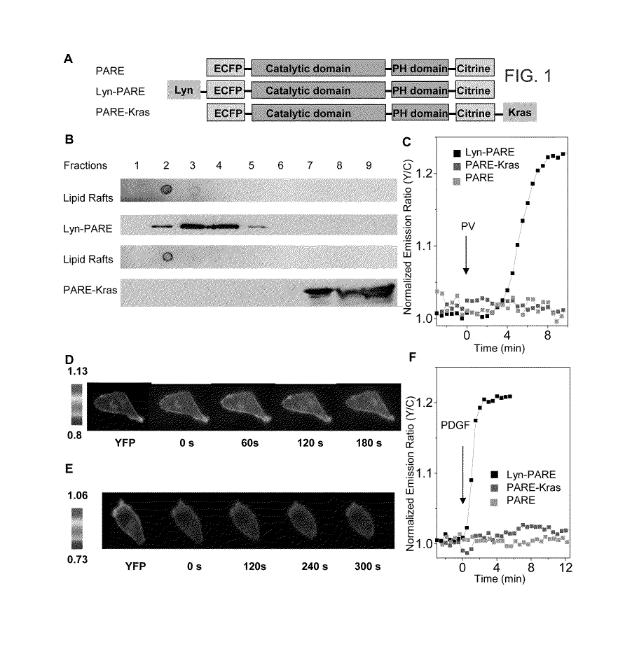

PDK1 activity reporters can be targeted to different plasma membrane microdomains. For example, the activation of two pools of PDK1 can be separately monitored by targeting a PDK1 activity reporter (e.g., PARE, described below) to lipid rafts and to non-lipid raft membranes. For example, the N-terminal portion of Lyn kinase can be used to direct a PDK1 activity reporter to lipid rafts through myristoylation and palmitoylation (FIG. 1A), while a C-terminal C(A)₂X (C stands for a cysteine, A for aliphatic amino acids, and X for any amino acid) sequence together with a polylysine motif can be used to anchor a PDK1 activity reporter to a non-raft plasma membrane.

PDK1 activity reporters can be constructed using components and methods described below and in the specific Examples. In some embodiments PDK1 is sandwiched between a RET pair. A “RET pair” contains a donor moiety and an acceptor moiety which exhibit a detectable resonance energy transfer when the donor moiety is excited.

Donor and Acceptor Moieties

As used here, a “donor moiety” is a fluorophore or a luminescent moiety. The absorption spectrum of the “acceptor moiety” overlaps the emission spectrum of the donor moiety. The acceptor moiety does not need to be fluorescent and can be a fluorophore, chromophore, or quencher. In some embodiments both the donor and acceptor moieties are fluorescent proteins. In other embodiments both the donor and acceptor moieties are luminescent moieties. In yet other embodiments, either one of the donor or acceptor moieties can be a fluorescent protein while the other moiety is a luminescent moiety. In other embodiments, the acceptor moiety is a “quencher moiety.”

When both the donor and acceptor moieties are fluorophores, resonance energy transfer is detected as fluorescence resonance energy transfer (FRET). If a luminescent moiety is involved, resonance energy transfer is detected as luminescent resonance energy transfer (LRET). LRET includes bioluminescent resonance energy transfer (BRET; Boute et al., Trends Pharmacol. Sci. 23, 351-54, 2002; Ayoub et al., J. Biol. Chem. 277, 21522-28, 2002). Because excitation of the donor moiety does not require exogenous illumination in an LRET method, such methods are particularly useful in live tissue and animal imaging, because penetration of the excitation light is no longer a concern. LRET methods have a high contrast and high signal-to-noise ratio; 2) no photobleaching occurs; and 3) quantification is simplified because the acceptor moiety is not directly excited.

Suitable acceptor moieties include, for example, a coumarin, a xanthene, a fluorescein, a fluorescent protein, a circularly permuted fluorescent protein, a rhodol, a rhodamine, a resorufin, a cyanine, a difluoroboradiazaindacene, a phthalocyanine, an indigo, a benzoquinone, an anthraquinone, an azo compound, a nitro compound, an indoaniline, a diphenylmethane, a triphenylmethane, and a zwitterionic azopyridinium compound.

Suitable donor moieties include, but are not limited to, a coumarin, a xanthene, a rhodol, a rhodamine, a resorufin, a cyanine dye, a bimane, an acridine, an isoindole, a dansyl dye, an aminophthalic hydrazide, an aminophthalimide, an aminonaphthalimide, an aminobenzofuran, an aminoquinoline, a dicyanohydroquinone, a semiconductor fluorescent nanocrystal, a fluorescent protein, a circularly permuted fluorescent protein, and fluorescent lanthanide chelate.

Fluorescent Proteins

In some preferred embodiments either or both of the donor and acceptor moieties is a fluorescent protein. Suitable fluorescent proteins include green fluorescent proteins (GFP), red fluorescent proteins (RFP), yellow fluorescent proteins (YFP), and cyan fluorescent proteins (CFP). Useful fluorescent proteins also include mutants and spectral variants of these proteins which retain the ability to fluoresce.

RFPs include Discosoma RFPs, such Discosoma DsRed (SEQ ID NO:8) or a mutant thereof which includes an Ile125Arg mutation, or a non-oligomerizing tandem DsRed containing, for example, two RFP monomers linked by a peptide linker For example, a non-oligomerizing tandem RFP can contain two DsRed monomers or two mutant DsRed-I125R monomers linked by a peptide.

Useful GFPs include an Aequorea GFP (SEQ ID NO:9), a Renilla GFP, a Phialidium GFP, and related fluorescent proteins for example, a cyan fluorescent protein (CFP), a yellow fluorescent protein (YFP), or a spectral variant of the CFP or YFP. CFP (cyan) and YFP (yellow) are color variants of GFP. CFP and YFP contain 6 and 4 mutations, respectively. They are Tyr66Try, Phe66Leu, Ser65Thr, Asn145Ile, Met153Thr, and Val163Ala in CFP and Ser65Gly, Val168Leu, Ser72Ala, and Thr203Tyr. Spectral variants include an enhanced GFP (EGFP; SEQ ID NO:10), an enhanced CFP (ECFP; SEQ ID NO:11), an enhanced YFP (EYFP; SEQ ID NO:12), and an EYFP with V68L and Q69K mutations. Other examples of fluorescent proteins comprising mutations are Aequorea GFP with one or more mutations at amino acid residues A206, L221 or F223 (e.g., mutations A206K, L221K, F223R, Q80R); mutations L221K and F223R of ECFP, and EYFP-V68L/Q69K of SEQ ID NO:9. See also US 2004/0180378; U.S. Pat. Nos. 6,150,176; 6,124,128; 6,077,707; 6,066,476; 5,998,204; and 5,777,079; Chalfie et al., Science 263:802-805, 1994. The truncated ECFP shown in SEQ ID NO:13 is especially useful.

Other useful GFP-related fluorescent proteins include those having one or more folding mutations, and fragments of the proteins that are fluorescent, for example, an A. victoria GFP from which the two N-terminal amino acid residues have been removed. Several of these fluorescent proteins contain different aromatic amino acids within the central chromophore and fluoresce at a distinctly shorter wavelength than the wild type GFP species. For example, the engineered GFP proteins designated P4 and P4-3 contain, in addition to other mutations, the substitution Y66H; and the engineered GFP proteins designated W2 and W7 contain, in addition to other mutations, Y66W.

Folding mutations in Aequorea GFP-related fluorescent proteins improve the ability of the fluorescent proteins to fold at higher temperatures and to be more fluorescent when expressed in mammalian cells, but have little or no effect on the peak wavelengths of excitation and emission. If desired, these mutations can be combined with additional mutations that influence the spectral properties of GFP to produce proteins with altered spectral and folding properties, and, particularly, with mutations that reduce or eliminate the propensity of the fluorescent proteins to oligomerize. Folding mutations, with respect to SEQ ID NO:11, include the substitutions F64L, V68L, S72A, T44A, F99S, Y145F, N1461, M153T, M153A, V163A, I167T, S175G, S205T, and N212K.

Luminescent Moieties

Luminescent moieties useful in PDK1 activity reporters include lanthanides, which can be in the form of a chelate, including a lanthanide complex containing the chelate (e.g, β-diketone chelates of cerium, praseodymium, neodymium, promethium, samarium, europium, gadolinium, terbium, dysprosium, holmium, erbium, thulium, or ytterbium). Lanthanide chelates are well known in the art. See Soini and Kojola, Clin. Chem. 29, 65, 1983; Hemmila et al., Anal. Biochem. 137, 335 1984; Lovgren et al., In: Collins & Hoh, eds., Alternative Immunoassays, Wiley, Chichester, U. K., p. 203, 1985; Hemmila, Scand. J. Clin. Lab. Invest. 48, 389, 1988; Mikola et al., Bioconjugate Chem. 6, 235, 1995; Peruski et al., J. Immunol. Methods 263, 35-41, 2002; U.S. Pat. No. 4,374,120; and U.S. Pat. No. 6,037,185. Suitable β-diketones are, for example, 2-naphthoyltrifluoroacetone (2-NTA), 1-naphthoyltrifluoroacetone (1-NTA), p-methoxybenzoyltrifluoroacetone (MO-BTA), p-fluorobenzoyltrifluoroacetone (F-BTA), benzoyltrifluoroacetone (BTA), furoyltrifluoroacetone (FTA), naphthoylfuroylmethane (NFM), dithenoylmethane (DTM), and dibenzoylmethane (DBM). See also US 20040146895.

Luminescent proteins include, but are not limited to, lux proteins (e.g., luxCDABE from Vibrio fischerii), luciferase proteins (e.g., firefly luciferase, Gaussia luciferase, Pleuromamma luciferase, and luciferase proteins of other beetles, Dinoflagellates (Gonylaulax; Pyrocystis;), Annelids (Dipocardia), Molluscs (Lativa), and Crustacea (Vargula; Cypridina), and green fluorescent proteins of bioluminescent coelenterates (e.g., Aequorea Victoria, Renilla mullerei, Renilla reniformis; see Prendergast et al., Biochemistry 17, 3448-53, 1978; Ward et al., Photochem. Photobiol. 27, 389-96, 1978; Ward et al., J. Biol. Chem. 254, 781-88, 1979; Ward et al., Photochem. Photobiol. Rev 4, 1-57, 1979; Ward et al., Biochemistry 21, 4535-40, 1982). Many of these proteins are commercially available. Firefly luciferase is available from Sigma, St. Louis, Mo., and Boehringer Mannheim Biochemicals, Indianapolis, Ind. Recombinantly produced firefly luciferase is available from Promega Corporation, Madison, Wis. Jellyfish aequorin and luciferase from Renilla are commercially available from Sealite Sciences, Bogart, Ga.

The DNA sequences of aequorin and other luciferases can be derived from a variety of sources. For example, cDNA can be prepared from mRNA isolated from the species disclosed above. See Faust, et al., Biochem. 18, 1106-19, 1979; De Wet et al., Proc. Natl. Acad. Sci. USA 82, 7870-73, 1985.

Luciferase substrates (luciferins) are well known and include coelenterazine (available from Molecular Probes, Eugene, Oreg.) and ENDUREN™. These cell-permeable reagents can be directly administered to cells, as is known in the art. Luciferin compounds can be prepared according to the methods disclosed by Hori et al., Biochemistry 14, 2371-76, 1975; Hori et al., Proc. Natl. Acad. Sci. USA 74, 4285-87, 1977).

Dark Quenchers

In some embodiments the acceptor moiety is a quencher moiety, preferably a “dark quencher” (or “black hole quencher”) as is known in the art. “Dark quenchers” themselves do not emit photons. Use of a “dark quencher” reduces or eliminates background fluorescence or luminescence which would otherwise occur as a result of energy transfer from the donor moiety. Suitable quencher moieties include dabcyl (4-(4′-dimethylaminophenylazo)-benzoic acid), QSY™-7 carboxylic acid, succinimidyl ester (N,N′-dimethyl-N,N′-diphenyl-4-((5-t-butoxycarbonylaminopentyl)aminocarbon yl) piperidinylsulfone-rhodamine (a diarylrhodamine derivative from Molecular Probes, Eugene, Oreg.). Suitable quencher moieties are disclosed, for example, in US 2005/0118619; US 20050112673; and US 20040146959.

Any suitable fluorophore may be used as the donor moiety provided its spectral properties are favorable for use with the chosen dark quencher. The donor moiety can be, for example, a Cy-dye, Texas Red, a Bodipy dye, or an Alexa dye. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, a fluorescein (e.g., fluorescein, tetrachlorofluorescein, hexachlorofluorescein), rhodamine, tetramethyl-rhodamine, or other like compound. Suitable fluorescent moieties for use with dark quenchers include xanthene dyes, such as fluorescein or rhodamine dyes, including 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N;N′-tetramethyl-6-carboxyrhodamine (TAMRA), 6-carboxy-X-rhodamine (ROX). Suitable fluorescent reporters also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS).

Other suitable fluorescent moieties include coumarins, such as 3-phenyl-7-isocyanatocoumarin; acridines, such as 9-isothiocyanatoacridin-e and acridine orange; N-(p-(2-benzoxazolyl)phenyl)maleimide; cyanines, such as indodicarbocyanine 3 (Cy3), indodicarbocyanine 5 (Cy5), indodicarbocyanine 5.5 (Cy5.5), 3-1-carboxy-pentyl)-3′-ethyl-5,5′-dimethyl-loxacarbocyanine (CyA); 1H,5H,1H,15H-Xantheno[2,3,4-ij:5,6,7-i′j′]diquinol-izin-18-ium, 9-[2(or 4)-[[[6-[2,5-dioxo-1-pyrrolidinyl)oxy]-6-oxohexyl]amino]sulfonyl]-4(or 2)-sulfophenyl]-2,3,6,7,12,13,16,17-octahyd-ro-inner salt (TR or Texas Red); BODIPY™ dyes; benzoxaazoles; stilbenes; pyrenes; and the like.

FRET-Based PDK1 Activity Reporters

In some embodiments a PDK1 activity reporter is a genetically-encoded, FRET-based, reversible reporter which comprises, from N to C terminus, (a) a first polypeptide comprising a polypeptide donor moiety of a fluorescence resonance energy transfer (FRET) pair; (b) a second polypeptide comprising PDK1, linked at its N terminus to the C terminus of the first polypeptide; and (c) a third polypeptide comprising a polypeptide acceptor moiety of the FRET pair, linked by its N terminus to the C terminus of the second polypeptide.

In some embodiments the polypeptide donor moiety is a cyan fluorescent protein, such as enhanced cyan fluorescent protein. In some embodiments and the polypeptide acceptor moiety is a yellow fluorescent protein, such as Citrine. In other embodiments the polypeptide donor moiety is a cyan fluorescent protein, such as enhanced cyan fluorescent protein and the polypeptide acceptor moiety is a yellow fluorescent protein, such as Citrine. One embodiment of a PDK1 activity reporter is “PDK1 Activation Reporter” (PARE), which contains full length PDK1 between the FRET pair ECFP and Citrine (FIG. 1A). FRET pairs of red and green fluorescent proteins also are useful.

Subcellular Targeting

Subcellular targeting sequences which can target a PDK1 activity reporter to a subcellular domain, such as a plasma membrane, a nuclear membrane, an endoplasmic reticulum, a mitochondria, a mitochondrial matrix, a chloroplast, a medial trans-Golgi cisternae, a lumen of a lysosome, or a lumen of an endosome, are well known in the art. Examples include the plasma membrane targeting sequence shown in SEQ ID NO:1, the nuclear localization signal sequence shown in SEQ ID NO:2, the mitochondrial localization sequence shown in SEQ ID NO:3, and the mitochondrial matrix targeting signal shown in SEQ ID NO:4. Targeting sequences can be linked to a reporter using, for example, a tetracysteine motif such as Cys Cys Xaa Xaa Cys Cys (SEQ ID NO:5).

A subcellular targeting sequence can be attached either at the N or the C terminus of a PDK1 activity reporter. In some embodiments the subcellular targeting sequence is a lipid raft targeting peptide sequence. In some embodiments the subcellular targeting sequence is a non-raft plasma membrane targeting peptide sequence. Examples of such targeting sequences are provided in Example 1.

### Nucleic Acid Molecules

In some embodiments, PDK1 activity reporters contain only protein components. Such fusion proteins can be expressed recombinantly, using nucleic acid molecules that encode a PDK1 activity reporter. A nucleic acid molecule encoding a PDK1 activity reporter can comprise any nucleotide sequence which encodes the amino acid sequence of the reporter. Nucleic acid molecules include single- and double-stranded DNA (including cDNA) and mRNA. Many kits for constructing fusion proteins are available from companies such as Promega Corporation (Madison, Wis.), Stratagene (La Jolla, Calif.), CLONTECH (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

In some embodiments the nucleic acid molecules are expression constructs which contain the necessary elements for the transcription and translation of an inserted coding sequence encoding a PDK1 activity reporter. Expression constructs can be used as vectors for introducing PDK1 activity reporters into cells. Methods which are well known to those skilled in the art can be used to construct expression vectors containing sequences encoding PDK1 activity reporters and appropriate transcriptional and translational control elements. These methods include in vitro recombinant DNA techniques, synthetic techniques, and in vivo genetic recombination. Such techniques are described, for example, in Sambrook et al. (1989) and in Ausubel et al., Current Protocols in Molecular Biology, John Wiley & Sons, New York, N.Y., 1989.

Expression vectors can be expressed in a variety of host cells. These include, but are not limited to, microorganisms, such as bacteria transformed with recombinant bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors (e.g., Ti or pBR322 plasmids), or animal cell systems, particularly mammalian systems, including human systems. See WO 01/98340, which is incorporated herein by reference in its entirety. The choice of vector components and appropriate host cells is well within the capabilities of those skilled in the art.

Other Methods of Constructing PDK1 Activity Reporters

Alternatively, protein or non-protein donor and/or acceptor moieties can be linked to a PDK1 polypeptide by covalent attachment. There are a variety of methods known in the art which are useful for this purpose. For example, the attachment can be direct, via a functional group on the polypeptide (e.g., amino, carboxyl and sulfhydryl groups) and a reactive group on the fluorophore. Free amino groups in the polypeptide can be reacted with fluorophores derivatized with isothiocyanate, maleic anhydride, N-hydroxysuccinimide, tetrafluorylphenyl and pentafluoryl esters. Free carboxyl groups in the polypeptide can be reacted with carbodiimides such as 1-ethyl-3-[dimethylaminopropyl]carbodiimide hydrochloride to create a reactive moiety that will react with an amine moiety on the donor or acceptor moiety. Sulfhydryl groups can be attached to donor or acceptor moieties modified with maleimide and iodoacetyl groups, although such linkages are more susceptible to reduction than linkages involving free amino groups. The polypeptide can also be linked indirectly via an intermediate linker or spacer group, using chemical groups such as those listed above.

It is also possible to produce PDK1 activity reporters using chemical methods to synthesize the amino acid sequence of the polypeptide and, optionally, one or more fluorescent or luminescent proteins. Methods include direct peptide synthesis using solid-phase techniques (Merrifield, J. Am. Chem. Soc. 85, 2149-2154, 1963; Roberge et al., Science 269, 202-204, 1995). Protein synthesis can be performed using manual techniques or by automation. Automated synthesis can be achieved, for example, using Applied Biosystems 431A Peptide Synthesizer (Perkin Elmer). Optionally, fragments of polypeptide portions of PDK1 activity reporters can be separately synthesized and combined using chemical methods to produce a full-length PDK1 activity reporter molecule. See WO 01/98340.

Delivery of Reporters to Cells or Lipid Bilayers

Assays described below can be carried out either with intact cells comprising a PDK1 activity reporter or with lipid bilayers (e.g., cell membrane preparations or synthetic cell membranes) that contain a PDK1 activity reporter. In some embodiments the lipid bilayer comprises a lipid raft. In some embodiments the lipid bilayer consists essentially of a lipid raft. In other embodiments the lipid bilayer comprises a non-raft plasma membrane. In other embodiments the lipid bilayer consists essentially of a non-raft plasma membrane.

PDK1 activity reporters can be introduced into cells in vitro using reversible permeabilization techniques. See U.S. Pat. No. 6,127,177; U.S. Pat. No. 6,902,931; Russo et al., Nature Biotechnology 15, 278-82, March 1997; Santangelo et al., Nucleic Acids Res. 32, 1-9, Apr. 14, 2004. For genetically encoded PDK1 activity reporters, an expression vector comprising a reporter-encoding nucleotide sequence can be transfected into any cell in vitro in which it is desired to monitor PDK1 activation or distribution. Any transfection method known in the art can be used, including, for example, including, but not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and DEAE- or calcium phosphate-mediated transfection.

Useful vectors and methods of delivering nucleic acids to cells in vivo are disclosed, for example, in U.S. Pat. No. 6,825,012; U.S. Pat. No. 6,878,549; U.S. Pat. No. 6,645,942; U.S. Pat. No. 6,692,737; U.S. Pat. No. 6,689,758; U.S. Pat. No. 6,669,935; and U.S. Pat. No. 6,821,957.

### Methods of Detecting PDK1 Activation

PDK1 activation can be detected by detecting conformational changes in a PDK1 activity reporter. Broadly, the methods involve detecting a change in resonance energy transfer (e.g., FRET) of a PDK1 activity reporter when the reporter is subjected to a condition that activates PDK1 or that suppresses its activation. Activation induces a conformational change that changes resonance energy transfer from the donor moiety to the acceptor moiety.

A change in resonance energy transfer can readily be detected using methods well known in the art. See, e.g., US 2005/0118619; US 2002/0137115; US 2003/0165920; US 2003/0186229; US 2004/0137479; US 2005/0026234; US 2005/0054573; US 2005/0118619; U.S. Pat. No. 6,773,885; U.S. Pat. No. 6,803,201; U.S. Pat. No. 6,818,420; Ayoub et al., 2002; Boute et al., 2002; Domin et al., Prog. Biomed. Optics and Imaging, Proc. SPIE, vol 5139, 2003, pp 238-242; Evellin et al., Methods Mol. Biol. 284, 259-70, 2004; Honda et al., Proc. Natl. Acad. Sci. USA 98, 437-42, Feb. 27, 2001; Honda et al., Methods Mol. Biol. 3, 27-44, 1005; Mongillo et al., Cir. Res. 95, 67-75, Jul. 9, 2004; Mongillo et al., Methods Mol. Biol. 307, 1-14, 2005; Nagai et al., Proc. Natl. Acad. Sci. USA 101, 10554-59, Jul. 20, 2004; Nikolaev et al., J. Biol. Chem. 279, 37215-18, 2004; Polit et al., Eur. J. Biochem. 270, 1413-23, 2003; Ponsioen et al., EMBO Rep. 5, 1176-80, 2004; Santangelo et al., Nucl. Acids Res. 32, 1-9, e-published Apr. 14, 2004; and Warrier et al., Am. J. Physiol. Cell Physiol. 289, C455-61, August 2005. Properties which can be detected as resonance energy transfer (RET) measurements include a molar extinction coefficient at an excitation wavelength, a quantum efficiency, an excitation spectrum, an emission spectrum, an excitation wavelength maximum, an emission wavelength maximum, a ratio of excitation amplitudes at two wavelengths, a ratio of emission amplitudes at two wavelengths, an excited state lifetime, anisotropy, a polarization of emitted light, resonance energy transfer, and a quenching of emission at a wavelength.

Reporters can be used in cell-free systems (e.g., in lipid bilayer preparations), in isolated cells (for example, in primary cell culture or a cell line) or in cells in situ (e.g., in an isolated tissue sample, an isolated whole organ, or in a mammal). Absolute activation levels can be detected by obtaining a RET measurement in the assay system and comparing it to a standard curve obtained in vitro.

In some embodiments, steady-state RET measurements are first obtained and then measurements are taken after addition of a test compound to the assay system. The effects of the test compounds on PDK1 activation can be monitored by monitoring RET (e.g., in drug-screening methods).

In some embodiments a PDK1 activity reporter is used to measure spatiotemporal PDK1 dynamics. In some embodiments, resonance energy transfer of the PDK1 activity reporter is detected at a first time point or place, a second resonance energy transfer of the PDK1 activity reporter is detected at a second time point or place, and the two resonance energy transfers are compared. A difference between the first and second resonance energy transfers reflects a change in spatiotemporal PDK1 dynamics. In some embodiments the resonance energy transfer is FRET.

All such methods can be carried out in the presence or absence of a test compound. For example, detection of aberrant PDK1 activation in a tissue sample can be used to diagnose the presence of tumor cells in the tissue sample, which is useful both for diagnostic purposes and for identifying an appropriate treatment regimen.

In other embodiments a test compound is assayed for its ability to inhibit activation of PDK1. A lipid raft comprising a PDK1 activity reporter is contacted with a test compound in the presence of an activator of PDK1, such as pervanadate, insulin, or a growth factor (e.g., PDGF, EGF, or IGF). Resonance energy transfer of the PDK1 activity reporter is measured and compared with the resonance energy transfer of the PDK1 activity reporter in the absence of the test compound. In some embodiments the resonance energy transfer is FRET. The lipid raft may be a membrane fragment or may be a synthetic lipid raft. In other embodiments the lipid raft is present within the plasma membrane of an intact cell.

Screening methods can be used to identify test compounds that may be useful in the treatment of hyperproliferative disorders (36). Such disorders include, but are not limited to, benign or malignant tumors of the colon, duodenum, prostate, breast, ovary, skin (e.g., melanoma), liver, pancreas, kidney, endometrium, oral mucosa, lung (e.g., small cell or non-small cell lung carcinoma), urinary tract (e.g., transitional or squamous cell urinary carcinoma); nervous system (e.g. neuroblastoma, glioma, astrocytoma), or blood (e.g. childhood acute leukemia, non-Hodgkin's lymphomas, chronic lymphocytic leukemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus).

Screening methods also can be used to identify test compounds that may be useful in the treatment of diabetes and insulin resistance (37). For example, a test compound that mimics PtdIns(3,4,5)P would enable PDK1 to activate Akt in tissues of diabetic subjects. Such a compound would be expected to promote glucose uptake, glycogen synthesis and protein synthesis in these patients. Similarly, a test compound that activates PDK1 may have similar effects.

### Test Compounds

Test compounds can be pharmacologic agents already known in the art to affect PDK1 activation or can be compounds previously unknown to have such an activity. Test compounds can be naturally occurring or designed in the laboratory. They can be isolated from microorganisms, animals, or plants, and can be produced recombinantly, or synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection.

High-Throughput Assays

High throughput assays, when combined with use of activity reporters, permit simple, fast, and convenient high-throughput reading of dynamic kinase activities with high spatiotemporal resolution. These methods complement, yet offer unique advantages over, existing methods, including purified target-based biochemical screens and end-point focused phenotypic screens. Activity-based screens can be combined with phenotypic screens (e.g., Clemons, Curr. Op. Chem. Biol. 8, 334-38, 2004) to provide direct measurement of dynamic cellular activities of defined targets or the activity of a signaling pathway. Compared to in vitro assays, living cells can be used as reaction vessels with targets of interest, cofactors, and regulators present at endogenous levels in their natural cellular environment, where spatiotemporal control of signaling activities can be specifically followed. With the complexity of live systems maintained, the quality of the screening process is increased, enabling discovery of compounds with unique mechanisms of action. Thus, the simple yet powerful high-throughput activity assays should find immediate application in high-throughput screens for pharmacological reagents and drug candidates, as well as in parallel tracking of multiple physiological and pharmacological events at subcellular locations in living cells in chemical and functional genomics studies.

Fluorescence activated cell sorting (FACS) is well-suited for use with high throughput methods. For example, emission ratios of yellow-to-cyan (cyan excitation) for individual cells are detected during the first sorting—not all cells will have the same emission ratio and a distribution for the whole population can be plotted; the cells can be stimulated to activate PDK1 in the absence or presence of other drugs; emission ratios of individual cells are detected again during the second sorting; the difference in emission ratios, usually presented as a shift in the distribution, will reflect the changes in PDK1 activation.

### Kits

Expression vectors encoding one or more PDK1 activity reporters can be provided in a kit. A kit may also provide all or a subset of reagents useful in carrying out an assay described above. The kits may comprise written instructions, in paper or electronic form, or a reference to an on-line set of instructions. The instructions may contain data against which the results determined using the kit can be compared. Containers which hold, e.g., an expression vector encoding a PDK1 activity reporter or the components of any given kit can vary. The kits may be divided into compartments or contain separate vessels for each component. The components may be mixed together or may be separated. Optional components of the kit include means for collecting, processing, and/or storing test samples.

Each patent, patent application, and reference cited in this disclosure is expressly incorporated herein by reference in its entirety. A more complete understanding can be obtained by reference to the specific examples, which are provided for purposes of illustration only and are not intended to limit the scope of this disclosure.

### Example 1

### Materials and Methods

Materials

PDGF, IGF-1, EGF, LY294002, H₂O₂, Na₃VO₄ and C2-ceramide were purchased from Sigma-Aldrich Co. Anti-Akt, phospho-Akt (T308), tubulin, and PTEN antibodies were obtained from Cell Signaling Technology, Inc, and GFP antibody from eBioscience, Inc. Horseradish peroxidase-conjugated cholera toxin subunit B was purchased from Molecular Probes, catalase from CalBiochem, and Lipofectamine 2000 from Invitrogen. Complete Protease Inhibitor Cocktail Tablets were obtained from Roche Applied Science.

Preparation of Pervanadate

Pervanadate was prepared as previously described (31). In brief, 25 μl of 500 mMNa₃VO₄ and 1 μl 30% (v/v) H₂O₂ were mixed in 574 μl PBS. After 5 min, catalase was added to release excess H₂O₂, which yielded 20 mM pervanadate.

Constructs

PARE was generated by sandwiching full-length PDK1 between a FRET pair, ECFP and Citrine. Lyn-PARE and PARE-Kras were generated by the addition of the N-terminal portion of the Lyn kinase gene (GCIKSKRKDKD; SEQ ID NO:6) at the 5′ end and a C(A)₂X tag (C is cysteine, A is an aliphatic amino acid, and X is any amino acid) at the 3′ end (KKKKKSKTKCVIM; SEQ ID NO:7) of PARE, respectively. Lyn-PARE R472/474A was generated by site-directed mutagenesis. PTEN A4 (fused with a C-terminal fluorescent protein) was targeted to raft and non-raft regions of plasma membrane with the same set of targeting motifs. All of the constructs were generated in a modified version of the mammalian expression vector pcDNA 3.

Cell Transfection and Imaging

Cells were plated on sterilized glass coverslips in 35 mm dishes and were grown to 40% confluency in medium at 37° C. with 5% CO₂, then transfected with Lipofectamine 2000. In the case of NIH 3T3 and HeLa cells, the cells were serum-starved for 24 h. For imaging, cells were washed with Hanks' balanced salt solution (HBSS) and imaged in the dark at room temperature. Images were acquired on a Zeiss Axiovert 200M microscope with a cooled charge-coupled device camera, as previously described (8). Dual-emission ratio imaging was performed with a 420DF20 excitation filter, a 450DRLP dichroic mirror, and two emission filters. For CFP and YFP, 475DF40 and 535DF25, respectively, were used. Exposure times were 50-500 ms. Images were taken every 30 s. Imaging data were analyzed with Metafluor 6.2 software (Universal Imaging, Downingtown, Pa.). Fluorescence images were background-corrected by deducting the background (regions with no cells) from the emission intensities of CFP or YFP. Traces were normalized by taking the emission ratio before addition of drugs as 1.

Western Blot Analysis

Cells were washed with ice-cold PBS and then lysed in RIPA lysis buffer containing protease inhibitor cocktail (Roche), 1 mM PMSF, 1 mM NaVO₄, 1 mM NaF and 25 nM calyculin A. Total cell lysates were incubated on ice for 30 min, then centrifuged at 4° C. for 20 min. Total protein was separated with 7.5% SDS-PAGE and transferred to nitrocellulose membranes. The membranes were blocked with TBS containing 0.05% Tween-20 and 1% bovine serum albumin, then incubated with primary antibodies overnight at 4° C. After incubation with the appropriate horseradish peroxidase-conjugated secondary antibodies, the bands were visualized by enhanced chemiluminescence. The intensity of the bands was quantified with ImageJ software.

Membrane Preparation

Cells were rinsed twice with ice-cold PBS and harvested into 10 mM Tris (pH 7.4) with 150 mM NaCl, 5 mM EDTA, 2 mM PMSF, 2 mM NaVO₄, 2 mM NaF, and 50 nM calyculin A, with protease inhibitor cocktail. The cells then were subjected to mechanical disruption with 15 strokes of a homogenizer. Homogenates were centrifuged at 2,300×g for 5 min at 4° C., and the supernatant was centrifuged at 18,000×g for 50 min at 4° C. (32). The resulting membrane pellets were resuspended in 10 mM Tris (pH 7.4) with 150 mM NaCl, 5 mM EDTA, 2 mM PMSF, 2 mM NaVO₄, 2 mM NaF and 50 nM calyculinA A, with protease inhibitor cocktail and 1% Triton X-100, for sucrose density gradient fractionation.

Sucrose Density Gradient Fractionation

Crude plasma membranes (or total cell lysates) were incubated in ice with periodic mixing for 1 h, then diluted 1:1 with 80% sucrose and layered on 4 ml 35% sucrose, followed by the addition of 1 ml 5% sucrose solution and 4.5 ml 10 mM Tris (pH 7.4) containing 150 mM NaCl and 5 mM EDTA. Ultracentrifugation was performed at 39,000×g for 18 h in a Beckman SW41-Ti rotor. All experimental steps were performed at 4° C. After ultracentrifugation, the top 3.5 ml of the sample was discarded. Nine 880-μl fractions were then collected, starting from the top of the gradient. The fractions were dot-blotted on nitrocellulose membranes and probed with HRP-conjugated cholera toxin subunit B antibody to identify the raft-containing fractions.

Differentiation of 3T3-L1 Adipocytes

3T3-L1 preadipocytes were grown to confluency in 10% calf serum/DMEM and stimulated with induction media (DMEM containing 10% fetal bovine serum, 1 μg/ml insulin, 1 μM dexamethasone, and 0.5 mM 3-isobutyl-1-methylxanthine) at 2 days post-confluency.

The medium was changed to insulin medium (1 μg/ml) 2 days after induction. Two days later, the medium was replaced with 10% fetal bovine serum/DMEM and then changed every 2 days. Full differentiation was achieved by 8 days after induction.

Glucose Uptake Assay

Adipocytes were incubated in Krebs-Ringer bicarbonate buffer supplemented with 30 mM HEPES, pH 7.4, with 0.5% BSA (KRBH/BSA) and 2.5 mM glucose for 3 h. The cells were washed once with PBS and incubated in BSA/KRH (25 mM HEPES-NaOH, pH 7.4, with 120 mM NaCl, 5 mM KCl, 1.2 mM MgSO₄, 1.3 mM CaCl₂, and 1.3 mM KH₂PO₄) without glucose for 15 min. The cells were then incubated with 100 nM insulin for 15 min. The assay was initiated by the addition of ^{[14C]}2-deoxyglucose (0.4 μCi/sample) and 5 mM glucose and terminated after 15 min by washing the cells three times with ice-cold PBS. Cells were solubilized in 1% Triton-100, and cell-associated radioactivity was determined by scintillation counting.

### Example 2

### The PDK1-Mediated Phosphorylation of the Activation Loop of Akt is Required for Akt Activity

Although the importance of PDK1 in the PI3K/Akt pathway has been established, the regulation of PDK1 has remained controversial (5). It has been suggested that PDK1 is constitutively active throughout the cell and cannot be further activated by growth factor stimulation (10). However, based on the observation that pervanadate (PV) can increase PDK1 activity at the plasma membrane, it has been suggested that PDK1 can be activated by growth factor stimulation in a similarly spatially controlled fashion (11).

We analyzed the activation of PDK1 in its native cellular context, the plasma membrane of living cells. More specifically, we examined different plasma membrane microdomains by generating a FRET-based PDK1 activation reporter (PARE) that could be targeted to these microdomains. To this end, we generated a construct in which the full-length PDK1 was sandwiched between a pair of fluorescent proteins, enhanced cyan fluorescent protein (ECFP) and Citrine, to allow us to monitor the conformational changes in PDK1 during its activation by changes in FRET. This reporter was targeted to membrane rafts using a targeting motif derived from Lyn kinase, and to non-raft regions of the plasma membrane using a targeting motif derived from Kras (8) (FIG. 1A). Sucrose density gradient fractionation experiments confirmed the localization of the two plasma membrane-targeted PDK1 activity reporters to the desired microdomains (FIG. 1B).

We then used these PDK1 activation reporters to examine the conformational changes associated with PV-induced activation of PDK1 in serum-starved NIH 3T3 cells. Interestingly, while no emission ratio change was observed in cells expressing the untargeted reporter, we saw a striking difference between the raft-targeted and non-raft-targeted PDK1 activity reporters: Only the raft-targeted PDK1 activity reporter showed an increase (of 20.7±3.3% [n=5]) in the yellow-to-cyan emission ratio, suggesting that the PV-induced activation of PDK1, which was previously reported to occur at the plasma membrane, instead occurs exclusively in raft microdomains of the plasma membrane (FIG. 1C).

We next examined growth factor-induced PDK1 activation. Serum-starved NIH 3T3 cells expressing one of the three variants of the PDK1 activity reporter were treated with 50 ng/ml platelet-derived growth factor (PDGF) and imaged. Strikingly, the raft-targeted PDK1 activity reporter showed an increase of 19±4% (n=7) in the yellow-to-cyan emission ratio upon growth factor stimulation, whereas no emission ratio change was observed in cells expressing the untargeted or non-raft-targeted PDK1 activity reporter (FIGS. 1D-1F). These data suggest that growth factor stimulation can induce the activation of PDK1 and that this activation also occurs exclusively in the raft microdomains of the plasma membrane.

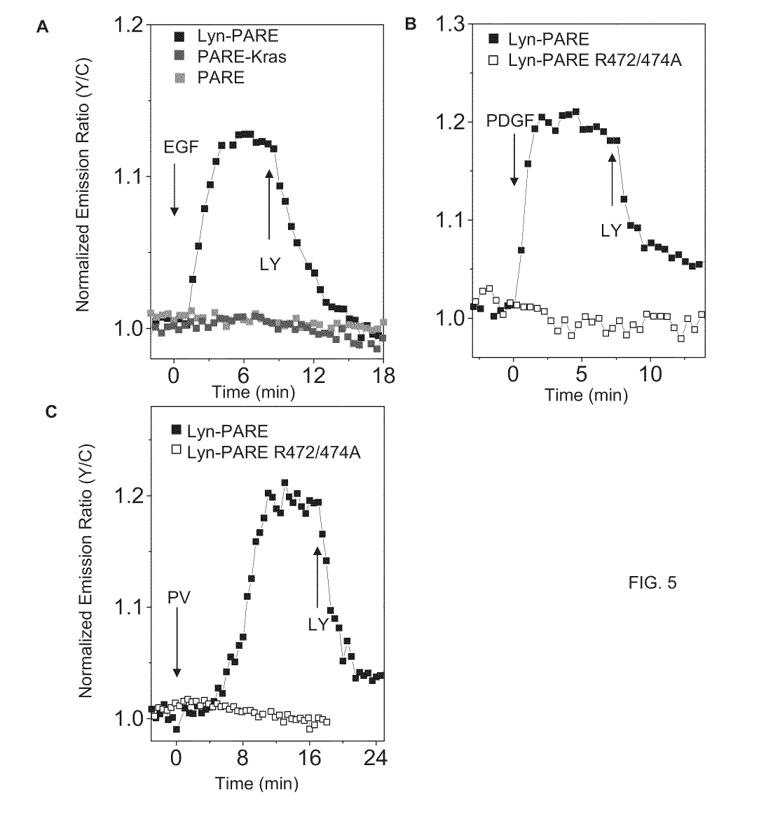

Using the lipid raft-targeted PDK1 activity reporter, we also examined the ability of other growth factors to activate PDK1 in live cells. Stimulation of serum-starved HeLa cells with 100 ng/ml epidermal growth factor (EGF) induced an emission ratio increase in the case of the raft-targeted PDK1 activity reporter (FIG. 5A). As was true for PDGF stimulation, the EGF-induced response was also restricted to the membrane raft microdomains (FIG. 5A), indicating that the differential activation pattern of PDK1 in the plasma membrane microdomains was not unique to NIH 3T3 cells or to PDGF receptor activation.

PDK1, like Akt, possesses a pleckstrin homology (PH) domain that binds to 3′ phosphoinositides, including PI(3,4,5)P₃ and PI(3,4)P₂. Studies have demonstrated that the phosphoinositide binding of PDK1 plays a crucial role in controlling the activation of Akt and its downstream signaling. For instance, PDK1^{K465E/K465E} knock-in mice expressing a mutant PDK1 that is incapable of binding to 3′ phosphoinositides are known to exhibit impaired growth and a marked insulin resistance and glucose intolerance (12). We therefore asked whether the observed PDK1 activation in response to PV or PDGF was dependent on its binding to 3′ phosphoinositides.

As shown in FIGS. 5B-C, mutation of the two Arg residues (R472/474) responsible for phosphoinositide binding to Ala (13) abolished PDK1 activation, as indicated by a lack of response of the raft-targeted PDK1 activity reporter variant. Furthermore, addition of the PI3K inhibitor LY294002 caused a reversal of the response of the PDK1 activity reporter after the PDGF-, EGF- or PV-stimulated emission ratio change was stabilized (FIG. 5A-5C), indicating that the response of the raft-targeted PDK1 activity reporter is reversible and is dependent on PI3K activity. The lack of change in the non-raft regions may reflect the inefficient activation of PDK1 in these regions. Alternatively, PDK1 may be locked into conformations that correspond to high PDK1 activity in these membrane micro domains.

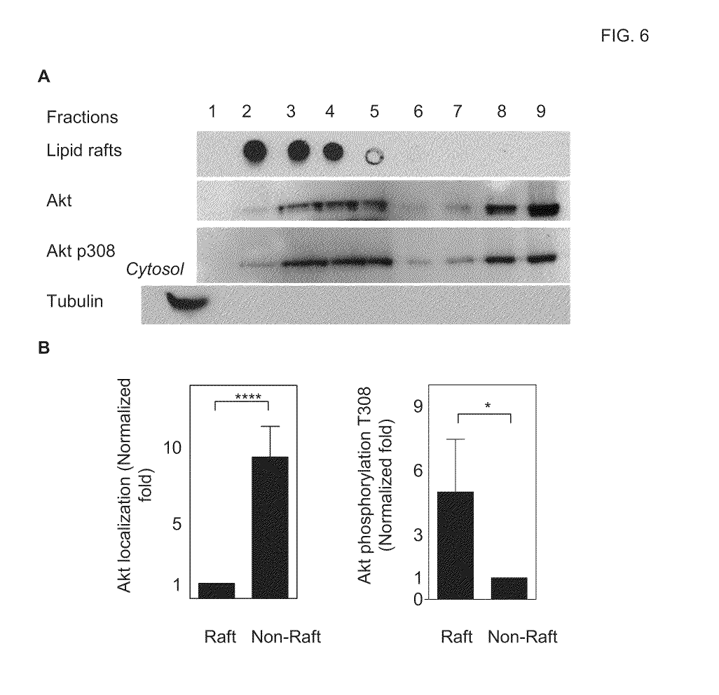

To directly examine endogenous PDK1 activity in these specific microdomains, we studied the phosphorylation of the natural substrate of PDK1, Akt, in raft and non-raft regions by sucrose density gradient separation followed by Western blot analysis. Phosphorylation of Akt by PDK1 at T308 was found to occur preferentially in the membrane rafts of insulin-stimulated HEK 293 cells, with 5.0±2.5-fold stronger phosphor Akt signal (normalized to the total Akt signal) in raft fractions than in non-raft fractions. This finding suggests that PDK1 is preferentially activated in membrane rafts (FIGS. 6A, 6B). In fact, considering the diffusion of activated PDK1 between membrane compartments, this 5-fold difference is likely an underestimation. Taken together, these studies suggest that PDK1 can be activated by a variety of stimulating signals, and this activation occurs in the raft microdomains of the plasma membrane.

### Example 3

### Localization of PTEN to Non-Raft Regions of the Plasma Membrane is Important for Proper Akt Signaling

We have previously shown that Akt activation, which is dependent on accumulation of 3′ phosphoinositides, is faster and stronger in membrane rafts (8). Our further demonstration of raft-specific activation of PDK1, also downstream of 3′ phosphoinositides, prompted us to examine the regulation of 3′ phosphoinositides in the context of plasma membrane microdomains. As a lipid phosphatase, PTEN plays an important role in restricting the accumulation of 3′ phosphoinosides. It has been shown to be largely cytosolic, with a small variable proportion associated with the plasma membrane (14). Although the membrane associated PTEN is the actual species that catalyze the degradation of 3′ phosphoinositides, its microdomain-specific membrane association and the importance of this membrane compartmentalization are not well understood.

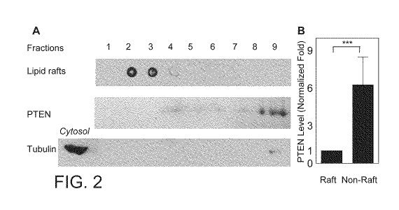

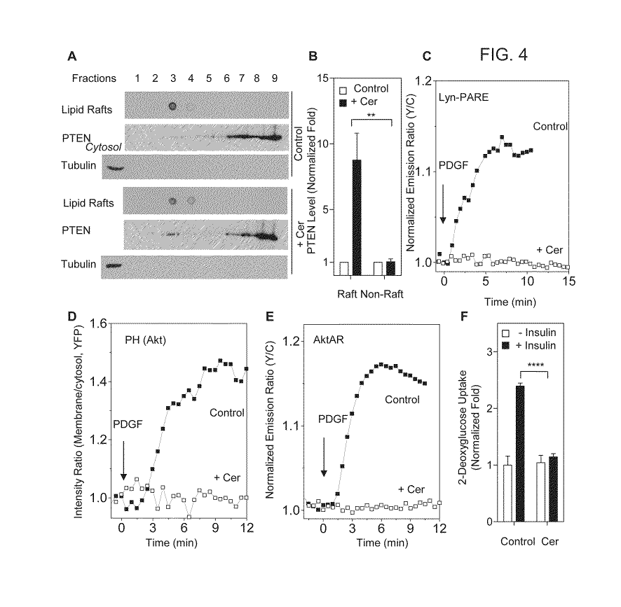

We first examined the membrane localization of endogenous PTEN by sucrose density gradient fractionation of crude plasma membranes isolated from HEK 293 cells. The majority of the membrane PTEN was found to reside in non-raft regions, with only a small amount being present in the membrane rafts (FIGS. 2A-B). The same pattern was also observed in 3T3-L1 adipocytes (FIG. 4A). These results indicated that PTEN primarily resides in the non-raft regions of the plasma membrane.

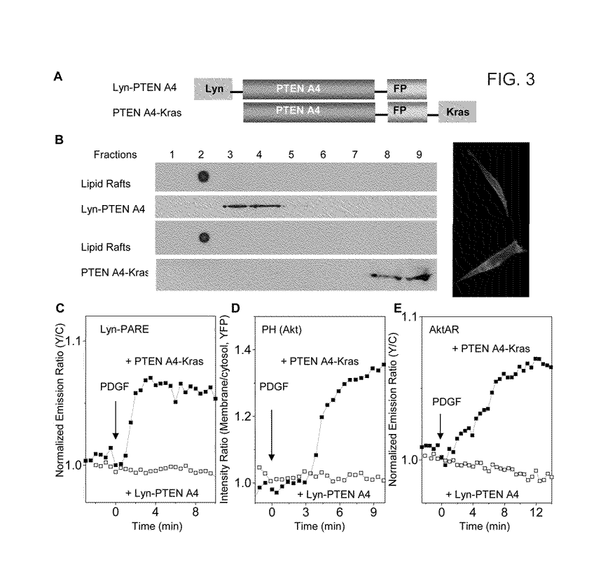

We then employed a genetic targeting approach to determine the role of membrane microdomain compartmentalization of PTEN in controlling downstream signaling. An active form of PTEN, PTEN A4 (15), has previously been described. In this form, four residues of the C-terminal tail region have been mutated to Ala to abolish the inhibitory phosphorylation. To alter the membrane microdomain localization of PTEN, we targeted PTEN A4 to raft microdomains with the Lyn-motif (FIG. 3A). As a control, a non-raft-targeted PTEN was also generated by attaching the Kras motif to PTEN A4 (FIG. 3A). The membrane localization of Lyn-PTEN A4 and PTEN A4-Kras was verified by sucrose density gradient fractionation (FIG. 3B).

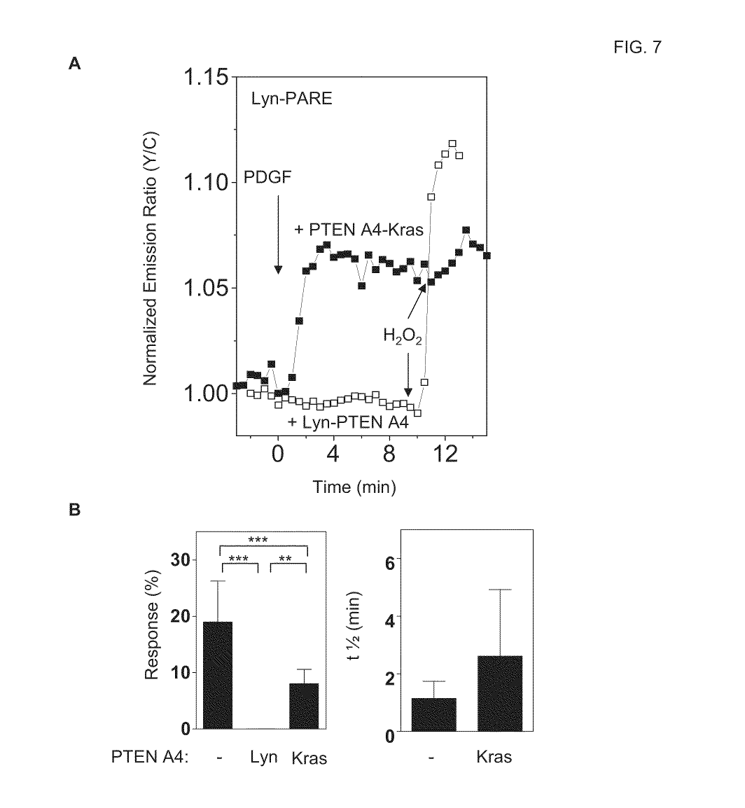

The effects of perturbing the membrane microdomain localization of PTEN were assessed at the levels of PDK1 activation, Akt recruitment, and Akt activity by using a series of fluorescent biosensors. First, we found that targeting of the active PTEN to membrane rafts via Lyn-PTEN A4 prevented growth factor-induced PDK1 activation, because no emission ratio change was observed in growth factor-stimulated NIH 3T3 cells co-expressing properly localized Lyn-PTEN A4 and the Lyn-PDK1 activity reporter (FIG. 3C). The presence of PTEN A4-Kras, on the other hand, did not prevent the activation of PDK1 in membrane rafts, although it did reduce the response of Lyn-PARE (FIG. 3C; FIG. 7A-B). The response to PDGF could be restored by treatment with 50 mM H₂O₂, a condition that has previously been shown to inhibit cellular PTEN (16) (FIG. 7A). This result further indicates that the suppression of raft PDK1 activation is caused by the high PTEN activity.

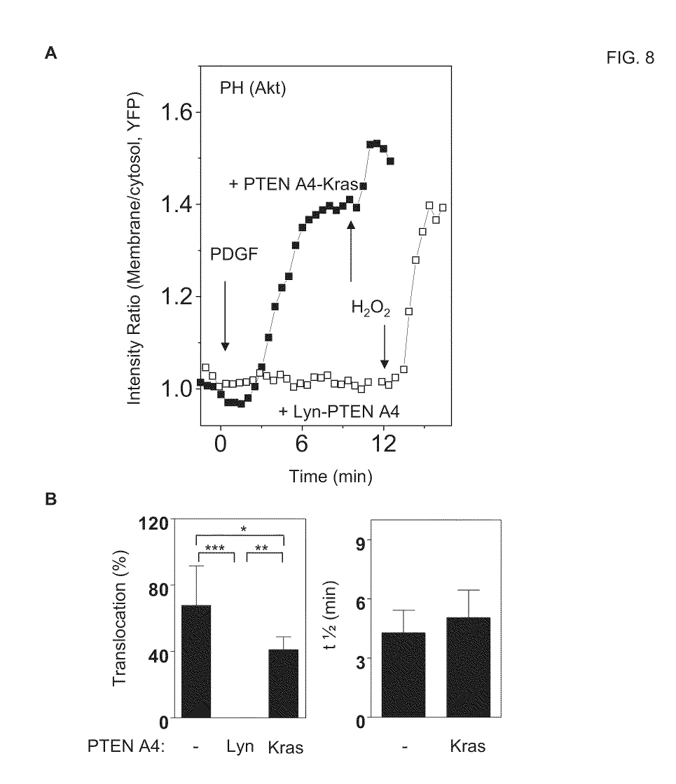

The strong inhibitory effect of Lyn-PTEN A4 on PDK1 activation could also be seen at the level of the membrane recruitment of Akt, as indicated by the lack of plasma membrane translocation of the YFP-tagged PH domain of Akt in response to growth factor stimulation (FIG. 3D). Inhibition of PTEN activity with 50 mM H₂O₂ restored the membrane translocation (FIG. 8A). In contrast, an active PTEN targeted to non-raft regions had a much lower inhibitory effect, producing a 40% inhibition of the translocation of Akt-PH when compared to cells without any expression of PTEN A4. This non-specific effect, presumably a result of the overexpression of an active PTEN, could also be reversed by H₂O₂ treatment (FIG. 3D; FIG. 8A-B).

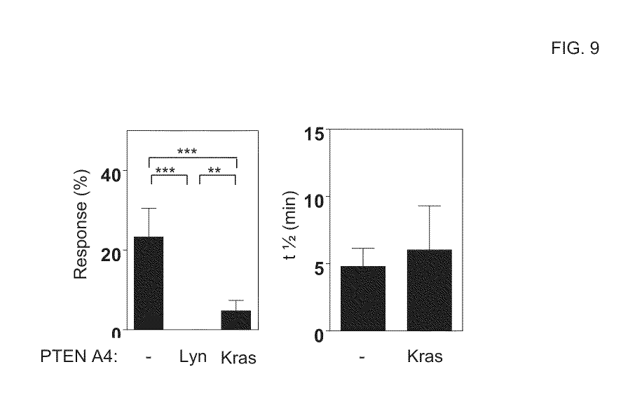

Finally, we used a previously developed FRET-based Akt activity reporter, AktAR (8), to probe the effect of perturbing membrane microdomain localization of PTEN on growth factor-stimulated Akt activity. This biosensor serves as a surrogate substrate for Akt and reports Akt activity by phosphorylation-dependent increases in FRET. As shown in FIG. 3E, the presence of Lyn-PTEN A4 abolished the response of AktAR to PDGF stimulation in NIH 3T3 cells, whereas cells expressing PTEN A4-Kras were still able to respond to PDGF stimulation (FIG. 3E; FIG. 9). These data demonstrate that preferential localization of PTEN outside membrane rafts has important functional consequence for maintaining PDK1 and Akt activity, and mis-localizing it to raft microdomains abolished downstream signaling.

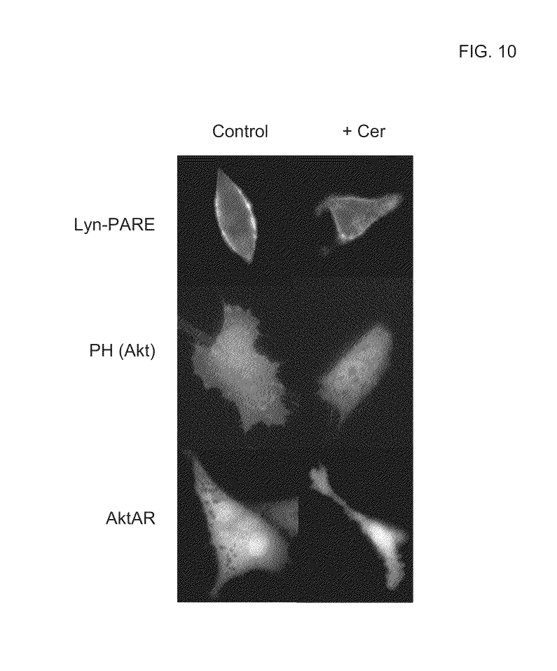

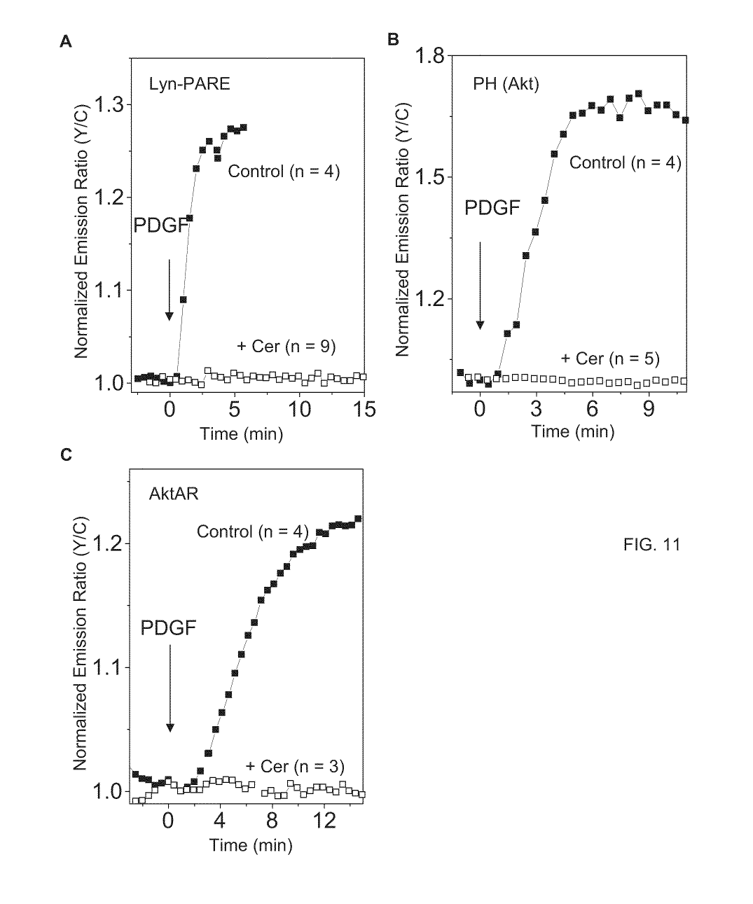

Given the importance of the specific membrane microdomain localization of PTEN in maintaining the activity of PI3K/Akt pathway, we postulated that dysregulation of this compartmentalization can disrupt cellular function and contribute to the development of pathologic conditions. Ceramide, a sphingolipid that is known to antagonize insulin action, has been suggested to be an important contributor to insulin resistance (17). The underlying mechanism is not clearly understood, but the major effect appears to be the inhibition of the PI3K/Akt pathway. Recently, ceramide was shown to translocate PTEN to caveolin-enriched microdomains in certain cell lines (18, 19). We confirmed this finding by showing that pre-incubation of 3T3-L1 adipocytes with C2-ceramide, a cell permeable ceramide analog, greatly increased the amount of PTEN found in raft fractions (8.8±2.1 fold, n=3), without changing the level of PTEN in non-raft regions of adipocyte membranes (FIGS. 4A-B). Importantly, treatment with C2-ceramide also blocked the PDGF-induced activation of PDK1 in membrane rafts and membrane recruitment of Akt, as well as Akt activity, in 3T3 μl preadipocytes and NIH 3T3 cells (FIGS. 4C-E; FIG. 10; FIG. 11). These findings are most consistent with a model in which ceramide suppresses PDK1 and Akt activity by specifically recruiting PTEN to lipid rafts. Furthermore, in agreement with previous reports (20), we found that this treatment also blocked insulin-induced glucose uptake in 3T3 L1 adipocytes (FIG. 4F), an important function mediated by the PI3K/Akt pathway, suggesting that ceramide-induced mislocalization of PTEN to membrane rafts can inhibit the functional output of the PI3K/Akt pathway.

Discussion

Activation of the PI3K/Akt pathway involves a series of tightly coupled molecular events occurring at the plasma membrane. However, how these molecular events are organized in the local signaling microdomains is not clear. By using a genetic targeting strategy in which the signaling events in specific membrane microdomains were monitored or perturbed, we have demonstrated that raft microdomains are critical for organizing the localization of active positive and negative regulators of the pathway. Dysregulation of this membrane compartmentalization undermined PI3K/Akt signaling transduction and may underlie pathological complications such as insulin resistance. As the kinase controlling the activation loop phosphorylation of Akt, PDK1 plays a critical role in PI3K/Akt signaling. Despite recent advances (21), the activation mechanisms of PDK1 are still not well understood. In the examples above, in contrast to the previously held belief that PDK1 is constitutively active and cannot be further activated by growth factor stimulation (10), we demonstrated that PDK1 can be activated by various growth factors and that this induced activation occurs in membrane rafts. Our genetic targeting approach allowed us to focus on specific events in specific plasma membrane domains of living cells, thereby enhancing the sensitivity of the assay and revealing previously unrecognized mechanistic details.

The mechanisms of this activation can be further elucidated, for example by examining Y373 phosphorylation, which has been shown to contribute to PV-induced PDK1 activation in the plasma membrane (11). This pool of further-activated PDK1 may directly contribute to the preferential activation of Akt in membrane rafts (8) and the proper functioning of the pathway. In addition, raft-activated PDK1 may play other important roles, since PDK1, as the master regulator of AGC kinase signal transduction(5), activates many other critical kinases, including protein kinase C (PKC), ribosomal S6 kinase (S6K), serum and glucocorticoid-inducible kinase (SGK), and p21-activated kinases (PAK). One study has showed that raft PDK1 recruits PKC and additional components to assemble a signaling complex that mediates NF-kB signaling in Jurkat cells stimulated with anti-CD3 and anti-CD28 (22).

PTEN is a critical negative regulator of the PI3K/Akt pathway. Our finding that membrane-associated PTEN is preferentially localized to non-raft regions is consistent with previous studies (23, 24), although in some cases raft PTEN has proved undetectable (25), possibly as a result of variations in the cell types and experimental conditions applied. The mechanism underlying this preferential localization to non-raft regions is not clear. One potential mediator is PI(4,5)P₂, a phosphoinositide that is critically involved in the membrane association of PTEN (26, 27). In the absence of protein binding, the polyunsaturated 2′-arachidonate chain of PI(4,5)P₂ does not favor partitioning into lipid rafts (7).

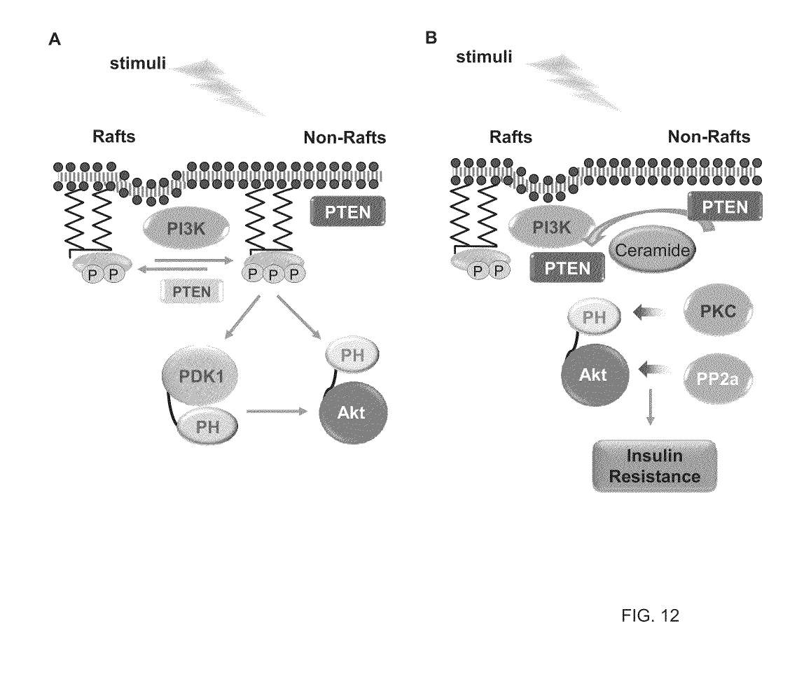

We further showed that the localization of PTEN to non-raft regions is critical to preserving the activity of PI3K/Akt pathway (FIG. 12A). Genetic targeting of PTEN A4 (an active form of PTEN) to membrane rafts completely abolished the activation of PDK1 in rafts as well as membrane recruitment of Akt and its activity. Non-raft-targeted PTENA4, on the other hand, showed inhibitory effects but did not prevent this pathway from being activated. The inhibition by non-raft-targeted PTEN A4 could have resulted from nonspecific effects of over-expressing an active PTEN. However, there is also a possibility that the enzyme molecules localized physically close to membrane rafts could directly interfere with signaling in raft microdomains.

Based on these new findings, we propose a model of membrane microdomain-mediated PI3K/Akt activation (FIG. 12A). Both the raft-specific activation of PDK1 and the lack of PTEN-mediated down regulation in these microdomains are believed to contribute to the preferential activation of Akt in membrane rafts (8). Yet our most striking finding was that perturbing this microdomain compartmentalization, either by genetic targeting of active PTEN to rafts or by ceramide-induced translocation of PTEN to the rafts, completely abolished the activity of the PI3K/Akt pathway. The previously observed inhibition of Akt recruitment upon raft disruption (7) is in line with this finding. Thus, plasma membrane microdomains can serve as platforms both for concentrating active signaling components such as activated PDK1 and Akt and for segregating them from non-raft associated negative regulators such as PTEN, thereby enabling the activation and functioning of the PI3K/Akt pathway. From a broader perspective, these data suggest that spatial compartmentalization not only defines the specificity and enhances the efficiency of signal transduction but also enables the activation and signaling mediated by critical pathways.

Dysregulation of this compartmentalization may occur under pathological conditions.

Ceramide is a lipid metabolite known to induce insulin resistance, and the underlying mechanisms are complex and not well understood (17). For example, ceramide has been shown to recruit the atypical PKC isoform PKCζ to lipid rafts, where PKCζ phosphorylates the PH domain of Akt and blocks its ability to interact with 3′ phosphoinositides (19, 28). On the other hand, ceramide has also been shown to promote the dephosphorylation of Akt by activating the Akt phosphatase protein phosphatase 2A (PP2A), one of the earliest known ceramide targets (29, 30). Here we propose that the ceramide-induced mis-localization of PTEN to membrane rafts critically contributes to the inhibitory effect of ceramide on PI3K/Akt signaling (FIG. 12B). In particular, the aforementioned mechanisms cannot account for the ceramide-mediated inhibition of the activation of raft PDK1. Furthermore, our genetic targeting of PTEN to membrane rafts recapitulated the strong inhibitory effects of ceramide on the activation of PDK1 and Akt. Together, these data suggest the membrane-microdomain compartmentalization is critical for maintaining proper PI3K/Akt signaling in response to insulin, and dysregulating raft-localized PI3K/Akt signaling by recruiting PTEN to these microdomains may be an underlying molecular mechanism for the insulin resistance caused by excess nutrients such as saturated fatty acids.

### REFERENCE LIST

1. Franke, T. F. (2008) PI3K/Akt: getting it right matters. Oncogene 27, 6473-6488.
2. Wong, K. K., Engelman, J. A. & Cantley, L. C. (2010) Targeting the PI3K signaling pathway in cancer. Current Opinion in Genetics & Development 20, 87-90.
3. Farese, R. V., Sajan, M. P. & Standaert, M. L. (2005) Insulin-sensitive protein kinases (atypical protein kinase C and protein kinase B/Akt): Actions and defects in obesity and Type II diabetes. Experimental Biology and Medicine 230, 593-605.
4. Bozulic, L. & Hemmings, B. A. (2009) PIKKing on PKB: regulation of PKB activity by phosphorylation. Curr. Opin. Cell Biol. 21, 256-261.
5. Mora, A., Komander, D., van Aalten, D. M. & Alessi, D. R. (2004) PDK1, the master regulator of AGC kinase signal transduction. Semin. Cell Dev. Biol. 15, 161-170.
6. Simons, K. & Toomre, D. (2000) Lipid rafts and signal transduction. Nat. Rev. Mol. Cell Biol. 1, 31-39.
7. Lasserre, R., Guo, X. J., Conchonaud, F., Hamon, Y., Hawchar, O., Bernard, A. M., Soudja, S. M., Lenne, P. F., Rigneault, H., Olive, D. et al. (2008) Raft nanodomains contribute to Akt/PKB plasma membrane recruitment and activation. Nat. Chem. Biol. 4, 538-547.
8. Gao, X. & Zhang, J. (2008) Spatiotemporal analysis of differential Akt regulation in plasma membrane microdomains. Mol. Biol. Cell 19, 4366-4373.
9. Hoeller, D., Volarevic, S. & Dikic, I. (2005) Compartmentalization of growth factor receptor signalling. Curr. Opin. Cell Biol. 17, 107-111.
10. Casamayor, A., Monice, N. A. & Alessi, D. R. (1999) Phosphorylation of Ser241 is essential for the activity of 3-phosphoinositide-dependent protein kinase-1: identification of five sites of phosphorylation in vivo. Biochem. J. 342 (Pt 2), 287-292.
11. Park, J., Hill, M. M., Hess, D., Brazil, D. P., Hofsteenge, J. & Hemmings, B. A. (2001) Identification of tyrosine phosphorylation sites on 3-phosphoinositide-dependent protein kinase-1 and their role in regulating kinase activity. Journal of Biological Chemistry 276, 37459-37471.
12. Bayascas, J. R., Wullschleger, S., Sakamoto, K., Garcia-Martinez, J. M., Clacher, C., Komander, D., van Aalten, D. M. F., Boini, K. M., Lan, F., Lipina, C. et al. (2008) Mutation of the PDK1 PH domain inhibits protein kinase B/Akt, leading to small size and insulin resistance. Molecular and Cellular Biology 28, 3258-3272.
13. Komander, D., Fairservice, A., Deak, M., Kular, G. S., Prescott, A. R., Downes, C. P., Safrany, S. T., Alessi, D. R. & van Aalten, D. M. F. (2004) Structural insights into the regulation of PDK1 by phosphoinositides and inositol phosphates. Embo Journal 23, 3918-3928.
14. Tamguney, T. & Stokoe, D. (2007) New insights into PTEN. J. Cell Sci. 120, 4071-4079.
15. Vazquez, F., Ramaswamy, S., Nakamura, N. & Sellers, W. R. (2000) Phosphorylation of the PTEN tail regulates protein stability and function. Molecular and Cellular Biology 20, 5010-5018.
16. Lee, S. R., Yang, K. S., Kwon, J., Lee, C., Jeong, W. & Rhee, S. G. (2002) Reversible inactivation of the tumor suppressor PTEN by H₂O₂. J. Biol. Chem. 277, 20336-20342.
17. Holland, W. L. & Summers, S. A. (2008) Sphingolipids, insulin resistance, and metabolic disease: new insights from in vivo manipulation of sphingolipid metabolism. Endocr. Rev. 29, 381-402.
18. Goswami, R., Singh, D., Phillips, G., Killcus, J. & Dawson, G. (2005) Ceramide regulation of the tumor suppressor phosphatase PTEN in rafts isolated from neurotumor cell lines. Journal of Neuroscience Research 81, 541-550.
19. Hajduch, E., Turban, S., Le Liepvre, X., Le Lay, S., Lipina, C., Dimopoulos, N., Dugail, I. & Hundal, H. S. (2008) Targeting of PKC zeta and PKB to caveolin-enriched microdomains represents a crucial step undeminning the disruption in PKB-directed signalling by ceramide. Biochemical Journal 410, 369-379.
20. Summers, S. A., Garza, L. A., Zhou, H. L. & Birnbaum, M. J. (1998) Regulation of insulin-stimulated glucose transporter GLUT4 translocation and Akt kinase activity by ceramide. Molecular and Cellular Biology 18, 5457-5464.
21. Masters, T. A., Calleja, V., Annoogum, D. A., Marsh, R. J., Applebee, C. J., Laguerre, M., Bain, A. J. & Larijani, B. (2010) Regulation of 3-phosphoinositidedependent protein kinase 1 activity by homodimerization in live cells. Sci. Signal. 3, ra78.
22. Lee, K. Y., D'Acquisto, F., Hayden, M. S., Shim, J. H. & Ghosh, S. (2005) PDK1 nucleates T cell receptor-induced signaling complex for NF-kappaB activation. Science 308, 114-118.
23. Caselli, A., Mazzinghi, B., Camici, G., Manao, G. & Ramponi, G. (2002) Some protein tyrosine phosphatases target in part to lipid rafts and interact with caveolin-1. Biochem. Biophys. Res. Comm. 296, 692-697.
24. Jahn, T., Leifheit, E., Gooch, S., Sindhu, S. & Weinberg, K. (2007) Lipid rafts are required for Kit survival and proliferation signals. Blood 110, 1739-1747.
25. Odriozola, L., Singh, G., Hoang, T. & Chan, A. M. (2007) Regulation of PTEN activity by its carboxyl-terminal autoinhibitory domain. Journal of Biological Chemistry 282, 23306-23315.
26. Walker, S. M., Leslie, N. R., Perera, N. M., Batty, I. H. & Downes, C. P. (2004) The tumour-suppressor function of PTEN requires an N-terminal lipid-binding motif Biochemical Journal 379, 301-307.
27. Iijima, M. & Devreotes, P. (2002) Tumor suppressor PTEN mediates sensing of chemoattractant gradients. Cell 109, 599-610.
28. Powell, D. J., Hajduch, E., Kular, G. & Hundal, H. S. (2003) Ceramide disables 3-phosphoinositide binding to the pleckstrin homology domain of protein kinase B (PKB)/Akt by a PKC zeta-dependent mechanism. Molecular and Cellular Biology 23, 7794-7808.
29. Chavez, J. A., Knotts, T. A., Wang, L. P., Li, G. B., Dobrowsky, R. T., Florant, G. L. & Summers, S. A. (2003) A role for ceramide, but not diacylglycerol, in the antagonism of insulin signal transduction by saturated fatty acids. Journal of Biological Chemistry 278, 10297-10303.
30. Dobrowsky, R. T., Kamibayashi, C., Mumby, M. C. & Hannun, Y. A. (1993) Ceramide Activates Heterotrimeric Protein Phosphatase-2A. Journal of Biological Chemistry 268, 15523-15530.
31. Huyer, G., Liu, S., Kelly, J., Moffat, J., Payette, P., Kennedy, B., Tsaprailis, G., Gresser, M. J. & Ramachandran, C. (1997) Mechanism of inhibition of protein-tyrosine phosphatases by vanadate and pervanadate. Journal of Biological Chemistry 272, 843-851.
32. Potier, S. M., Percherancier, Y., Galandrin, S., Breit, A., Gales, C. & Bouvier, M. (2008) Cholesterol-dependent Separation of the beta(2)-Adrenergic Receptor from Its Partners Determines Signaling Efficacy INSIGHT INTO NANOSCALE ORGANIZATION OF SIGNAL TRANSDUCTION. Journal of Biological Chemistry 283, 24659-24672.
33. Oudit et al., The role of phosphoinositide-3 kinase and PTEN in cardiovascular physiology and disease. J. Mol. Cell Cardiol. 37, 449-71, 2004.
34. Ananthanarayanan et al., Live-cell molecular analysis of Akt activation reveals roles for activation loop phosphorylation. J. Biol. Chem. 282, 36634-41, 2007.
35. Garcia-Echeverria & Sellers, Drug discovery approaches targeting the PI3K/Akt pathway in cancer. Oncogene 27, 5511-26, 2008.
36. Peifer C, Alessi DR. Small-molecule inhibitors of PDK1. ChemMedChem. 2008 December; 3(12):1810-38.
37. Alessi DR. Discovery of PDK1, one of the missing links in insulin signal transduction. Colworth Medal Lecture. Biochem Soc Trans. 2001 May; 29(Pt 2):1-14.

1. A phosphoinositide-dependent kinase 1 (PDK1) reporter, wherein the PDK1 activity reporter comprises:
(a) a first polypeptide comprising a polypeptide donor moiety of a fluorescence resonance energy transfer (FRET) pair;
(b) a second polypeptide comprising PDK1, wherein the N terminus of the second polypeptide is linked to the C terminus of the first polypeptide; and
(c) a third polypeptide comprising a polypeptide acceptor moiety of the FRET pair, wherein the N terminus of the third polypeptide is linked to the C terminus of the second polypeptide and wherein the polypeptide donor moiety and the polypeptide acceptor moiety exhibit a detectable resonance energy transfer when the donor moiety is excited.

2. The PDK1 activity reporter of claim 1 wherein the polypeptide donor moiety is a cyan fluorescent protein.

3. The PDK1 reporter of claim 1 wherein the polypeptide donor moiety is enhanced cyan fluorescent protein.

4. The PDK1 reporter of claim 2 wherein the polypeptide acceptor moiety is a yellow fluorescent protein.

5. The PDK1 reporter of claim 3 wherein the polypeptide acceptor moiety is Citrine.

6. The PDK1 reporter of claim 1 wherein the polypeptide acceptor moiety is a yellow fluorescent protein.

7. The PDK1 reporter of claim 1 wherein the polypeptide acceptor moiety is Citrine.

8. The PDK1 reporter of claim 1 further comprising a subcellular targeting peptide sequence.

9. The PDK1 reporter of claim 1 further comprising a lipid raft targeting peptide sequence.

10. The PDK1 reporter of claim 1 further comprising a lipid raft targeting peptide sequence comprising the amino acid sequence SEQ ID NO:6.

11. The PDK1 reporter of claim 1 further comprising a non-raft plasma membrane targeting peptide sequence.

12. The PDK1 reporter of claim 1 further comprising a non-raft plasma membrane targeting peptide sequence comprising the amino acid sequence SEQ ID NO:7.

13. A nucleic acid molecule encoding the PDK1 reporter of claim 1.

14. The nucleic acid molecule of claim 13 which is contained in an expression vector.

15. A lipid bilayer comprising the PDK1 reporter of claim 1.

16. The lipid bilayer of claim 15 which comprises a lipid raft.

17. The lipid bilayer of claim 15 which consists essentially of a lipid raft.

18. The lipid bilayer of claim 15 which comprises a non-raft plasma membrane.

19. The lipid bilayer of claim 15 which consists essentially of a non-raft plasma membrane.

20. The lipid bilayer of claim 15 which is in a cell membrane.

21. A cell comprising the nucleic acid molecule of claim 13.

22. A container comprising the nucleic acid molecule of claim 13.

23. A method for measuring spatiotemporal PDK1 dynamics, comprising:
(i) detecting a first FRET of the PDK1 activity reporter of claim 1 at a first time point or place;
(ii) detecting a second FRET of the PDK1 activity reporter at a second time point or place; and
(iii) comparing the first and the second FRETs,
wherein a difference between the first and the second FRETs reflects a change in spatiotemporal PDK1 dynamics.

24. The method of claim 23 further comprising contacting the PDK1 reporter with a test compound.

25. A method of assaying a test compound for its ability to inhibit activation of PDK1, comprising:
(i) contacting a lipid raft comprising the PDK1 activity reporter of claim 1 with a test compound in the presence of an activator of PDK1; and
(ii) assessing whether the test compound decreases FRET of the PDK1 activity reporter in the presence of the test compound in comparison to FRET of the PDK1 activity reporter in the absence of the test compound.

26. The method of claim 25 wherein the lipid raft is in a plasma cell membrane.

27. A kit comprising:
(i) a container comprising an expression vector encoding the PDK1 activity reporter of claim 1; and
(ii) instructions for using the PDK1 activity reporter for detecting PDK1 activation or detecting inhibition of PDK1 activation using FRET.


SEQUENCE LISTING (15 sequences)

SEQ ID NO: 1 - protein, 13 aa, Artificial Sequence
  plasma membrane targeting sequence
KKKKKSKTKCVIM

SEQ ID NO: 2 - protein, 10 aa, Artificial Sequence
  nuclear localization signal sequence
PKKKRKVEDA

SEQ ID NO: 3 - protein, 30 aa, Artificial Sequence
  mitochondrial localization signal sequence
MAIQLRSLFPLALPGMLALLGWWWFFSRKK

SEQ ID NO: 4 - protein, 16 aa, Artificial Sequence
  the mitochondrial matrix targeting signal
MLSLRGSIRFFKRSGI

SEQ ID NO: 5 - protein, 6 aa, Artificial Sequence
  tetracysteine motif
CCXXCC

SEQ ID NO: 6 - protein, 11 aa, Artificial Sequence
  N-terminal portion of the Lyn kinase gene
GCIKSKRKDKD

SEQ ID NO: 7 - protein, 13 aa, Artificial Sequence
  C(A)2X tag
KKKKKSKTKCVIM

SEQ ID NO: 8 - protein, 225 aa, Discosoma
MRSSKNVIKEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVVTVTQDSSLQDGCFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERTEGRHHLFL

SEQ ID NO: 9 - protein, 238 aa, Aequorea
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 10 - protein, 239 aa, Artificial Sequence
  enhanced GFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 11 - protein, 239 aa, Artificial Sequence
  enhanced CFP
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAAGITLGMDELYK

SEQ ID NO: 12 - protein, 239 aa, Artificial Sequence
  enhanced YFP
MVSKGEELFTGVVPILVELDGDVNGHKFSVSGEGEGDATYGKLTLKFICTTGKLPVPWPTLVTTFGYGLKCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 13 - protein, 228 aa, Artificial Sequence
  truncated enhanced CFP
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAA

SEQ ID NO: 14 - protein, 1039 aa, Artificial Sequence
  PDK1activation reporter
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSKLSKDPNEKRDHMVLLEFVTAAGITHGMDELYKAGTMARTTSQLYDAVPIQSSVVLCSCPSPSMVRTQTESSTPPGIPGGSRQGPAMDGTAAEPRPGAGSLQHAQPPPQPRKKRPEDFKFGKILGEGSFSTVVLARELATSREYAIKILEKRHIIKENKVPYVTRERDVMSRLDHPFFVKLYFTFQDDEKLYFGLSYAKNGELLKYIRKIGSFDETCTRFYTAEIVSALEYLHGKGIIHRDLKPENILLNEDMHIQITDFGTAKVLSPESKQARANSFVGTAQYVSPELLTEKSACKSSDLWALGCIIYQLVAGLPPFRAGNEYLIFQKIIKLEYDFPEKFFPKARDLVEKLLVLDATKRLGCEEMEGYGPLKAHPFFESVTWENLHQQTPPKLTAYLPAMSEDDEDCYGNYDNLLSQFGCMQVSSSSSSHSLSASDTGLPQRSGSNIEQYIHDLDSNSFELDLQFSEDEKRLLLEKQAGGNPWHQFVENNLILKMGPVDKRKGLFARRRQLLLTEGPHLYYVDPVNKVLKGEIPWSQELRPEAKNFKTFFVHTPNRTYYLMDPSGNAHKWCRKIQEVWRQRYQSHPDAAVQRSMVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSKLSKDPNEKRDHMVLLEFVTAAGITLGMDELYK

SEQ ID NO: 15 - DNA, 3120 bp, Artificial Sequence
  PDK1activation reporter
atggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactcacggcatggacgagctgtacaaggctggtaccatggccaggaccaccagccagctgtatgacgccgtgcccatccagtccagcgtggtgttatgttcctgcccatccccatcaatggtgaggacccagactgagtccagcacgccccctggcattcctggtggcagcaggcagggccccgccatggacggcactgcagccgagcctcggcccggcgccggctccctgcagcatgcccagcctccgccgcagcctcggaagaagcggcctgaggacttcaagtttgggaaaatccttggggaaggctctttttccacggttgtcctggctcgagaactggcaacctccagagaatatgcgattaaaattctggagaagcgacatatcataaaagagaacaaggtcccctatgtaaccagagagcgggatgtcatgtcgcgcctggatcaccccttctttgttaagctttacttcacatttcaggacgacgagaagctgtatttcggccttagttatgccaaaaatggagaactacttaaatatattcgcaaaatcggttcattcgatgagacctgtacccgattttacacggctgagattgtgtctgctttagagtacttgcacggcaagggcatcattcacagggaccttaaaccggaaaacattttgttaaatgaagatatgcacatccagatcacagattttggaacagcaaaagtcttatccccagagagcaaacaagccagggccaactcattcgtgggaacagcgcagtacgtttctccagagctgctcacggagaagtccgcctgtaagagttcagacctttgggctcttggatgcataatataccagcttgtggcaggactcccaccattccgagctggaaacgagtatcttatatttcagaagatcattaagttggaatatgactttccagaaaaattcttccctaaggcaagagacctcgtggagaaacttttggttttagatgccacaaagcggttaggctgtgaggaaatggaaggatacggacctcttaaagcacacccgttcttcgagtccgtcacgtgggagaacctgcaccagcagacgcctccgaagctcaccgcttacctgccggctatgtcggaagacgacgaggactgctatggcaattatgacaatctcctgagccagtttggctgcatgcaggtgtcttcgtcctcctcctcacactccctgtcagcctccgacacgggcctgccccagaggtcaggcagcaacatagagcagtacattcacgatctggactcgaactcctttgaactggacttacagttttccgaagatgagaagaggttgttgttggagaagcaggctggcggaaacccttggcaccagtttgtagaaaataatttaatactaaagatgggcccagtggataagcggaagggtttatttgcaagacgacgacagctgttgctcacagaaggaccacatttatattatgtggatcctgtcaacaaagttctgaaaggtgaaattccttggtcacaagaacttcgaccagaggccaagaattttaaaactttctttgtccacacgcctaacaggacgtattatctgatggaccccagcgggaacgcacacaagtggtgcaggaagatccaggaggtttggaggcagcgataccagagccacccggacgccgctgtgcagagatctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaa